(19) United States
(12) Patent Application Publication
(10) Pub. No.: US 2023/0184710 A1
(43) Pub. Date: Jun. 15, 2023

(54) NONENZYMATIC BIOSENSOR BASED ON METAL-MODIFIED POROUS BORON-DOPED DIAMOND ELECTRODE, AND METHOD FOR PREPARING SAME AND USE THEREOF

(71) Applicant: CENTRAL SOUTH UNIVERSITY, Changsha (CN)

(72) Inventors: Qiuping WEI, Changsha (CN); Li MA, Changsha (CN); Kechao ZHOU, Changsha (CN); Sichao ZENG, Changsha (CN); Ruitong ZHU, Changsha (CN); Wanlin YANG, Changsha (CN)

(73) Assignee: CENTRAL SOUTH UNIVERSITY, Changsha (CN)

(21) Appl. No.: 17/924,683

(22) PCT Filed: May 10, 2021
(86) PCT No.: PCT/CN2021/092645

§ 371 Date: Nov. 11, 2022

(30) Foreign Application Priority Data
May 11, 2020 (CN) 202010390541.5

(51) Int. Cl. G01N 27/30; G01N 27/327; G01N 33/487

(52) U.S. Cl. CPC G01N 27/308; G01N 27/3278; G01N 33/48714; G01N 33/48785; B82Y 40/00

(57) ABSTRACT

A nonenzymatic biosensor based on a metal-modified porous boron-doped diamond electrode, and a method for preparing the same and use thereof are provided. A working electrode of the nonenzymatic biosensor is a metal-modified porous boron-doped diamond electrode including a silicon wafer substrate and an electrode working layer arranged on a surface thereof, the electrode working layer is a porous boron-doped diamond layer modified with metal nanoparticles, and a pore surface of the porous boron-doped diamond layer contains an sp² phase. In the present invention, by combining chemical vapor deposition and magnetron sputtering and by means of a tubular atmosphere annealing furnace and an electrochemical workstation, the preparation of a multi-metal-modified porous boron-doped diamond composite electrode is realized. The electrode has the characteristics of high sensitivity, stability, and resolution, and can be widely used in the fields of the construction of electrochemical biosensors, the detection of heavy metals, etc.

### CROSS REFERENCE TO THE RELATED APPLICATIONS

This application is the national phase entry of International Application No. PCT/CN2021/092645, filed on May 10, 2021, which is based upon and claims priority to Chinese Patent Application No. 202010390541.5, filed on May 11, 2020, the entire contents of which are incorporated herein by reference.

### TECHNICAL FIELD

The present invention relates to a nonenzymatic biosensor based on a metal-modified porous boron-doped diamond electrode, and a method for preparing the same and use thereof, belonging to the technical field of preparation of nonenzymatic biosensors.

### BACKGROUND

A biosensor, as an instrument or apparatus that organically combines bioactive materials (enzymes, proteins, DNAs, antibodies, antigens, biomembranes, etc.) with physical transducers, is an advanced detection and monitoring method indispensable to the development of biotechnology, and is also a rapid microanalysis method at the molecular level of substances. According to the definition, the structure (composition) of the biosensor includes the following two parts: 1: bioactive materials (i.e. biosensitive membranes and molecular recognition elements): and 2: physical transducers (i.e. sensors). This patent relates to the sensors which are used for converting various biological, chemical and physical information into electrical signals. The information generated by biological reaction processes is diversified, and modern achievements of the microelectronics and sensing technology provide rich means for detecting the information, so that researchers have enough leeway to choose transducers when designing biosensors.

Enzymatic sensors are easily affected by factors such as environmental pH, temperature and humidity due to the restriction of enzymatic biochemical properties. During the preparation, packaging, transportation and storage of the enzymatic sensors, there will be inevitable risks of exposure to thermal deformation and chemical deformation, which brings quality control and production cost problems to the commercialization of the enzymatic sensors. In addition, the degree of immobilization of the enzymatic sensors will greatly affect the use performance of the sensors. Although there are many enzyme immobilization methods at present, including direct adsorption, sol-gel encapsulation, cross-linking, etc, for large-scale production and commercialization of the enzymatic sensor immobilization technology, a simple and reusable enzyme immobilization method is still the biggest research difficulty at present. Compared with the enzymatic sensors, nonenzymatic sensors are simple and controllable during immobilization, are suitable for large-scale sound fields, and have higher stability during use. However, the catalytic activities of the nonenzymatic sensors for different substances to be detected are different, so it is necessary to artificially perform specific accurate adjustment and control on sensitive materials modified on the nonenzymatic sensors, so as to obtain good selectivity and practical performance.

### SUMMARY

In view of the defects in the prior art, a first objective of the present invention is to provide a nonenzymatic biosensor based on a metal-modified porous boron-doped diamond electrode.

A second objective of the present invention is to provide a method for preparing the nonenzymatic biosensor based on a metal-modified porous boron-doped diamond electrode.

A third objective of the present invention is to provide use of the nonenzymatic biosensor based on a metal-modified porous boron-doped diamond electrode.

In the nonenzymatic biosensor based on a metal-modified porous boron-doped diamond electrode, provided by the present invention, a working electrode of the nonenzymatic biosensor is a metal-modified porous boron-doped diamond electrode including a silicon wafer substrate and an electrode working layer; and the electrode working layer is arranged on a surface of the silicon wafer substrate, the electrode working layer is a porous boron-doped diamond layer, the surface of which is modified with metal nanoparticles, and a pore surface of the porous boron-doped diamond layer contains an sp² phase.

The inventors found that by retaining the generated sp² phase on the pore surface of the porous boron-doped diamond layer, on the one hand, it is beneficial to reduce the interface resistance and improve the interface charge transfer, and a faster charge transfer rate means a higher electrochemical reaction rate, so the detection sensitivity and linear range of the sensor can be increased; and on the other hand, the adhesion with the metal nanoparticles can be increased, thereby further improving the stability of the electrode.

In the nonenzymatic biosensor based on a metal-modified porous boron-doped diamond electrode, provided by the present invention, a thickness of the porous boron-doped diamond layer is 5-20 μm, a grain size is 5-20 μm, and a crystal surface (111) is an exposed surface. Since the crystal surface (111) of the boron-doped diamond is pyramidal macroscopically, controlling the crystal surface (111) as the exposed surface can have a larger specific surface area and a higher intrinsic catalytic activity.

In the nonenzymatic biosensor based on a metal-modified porous boron-doped diamond electrode, provided by the present invention, particle sizes of the metal nanoparticles are 20-30 nm. When the particle sizes of the metal nanoparticles are controlled to be 20-30 nm, the catalytic activity of the obtained electrode is the highest.

In the nonenzymatic biosensor based on a metal-modified porous boron-doped diamond electrode, provided by the present invention, the metal nanoparticles are selected from at least one of gold, platinum, nickel and copper nanoparticles.

Preferably, in the nonenzymatic biosensor based on a metal-modified porous boron-doped diamond electrode, provided by the present invention, the metal nanoparticles are selected from gold and nickel, and according to an atomic ratio, gold:nickel=2:8. The inventors found that when the metal nanoparticles are selected from gold and nickel and gold:nickel=2:8, the catalytic activity of the obtained electrode is the highest.

Preferably, in the nonenzymatic biosensor based on a metal-modified porous boron-doped diamond electrode, provided by the present invention, the metal nanoparticles are selected from gold and platinum, and according to an atomic ratio, gold:platinum=1:1 The inventors found that when the metal nanoparticles are selected from gold and platinum and gold:platinum=1:1, the catalytic activity of the obtained electrode is the highest.

Preferably, in the nonenzymatic biosensor based on a metal-modified porous boron-doped diamond electrode, provided by the present invention, the metal nanoparticles are selected from nickel and copper, and according to an atomic ratio, nickel:copper=6:4. The inventors found that when the metal nanoparticles are selected from nickel and copper and nickel:copper=6:4, the catalytic activity of the obtained electrode is the highest.

The method for preparing the nonenzymatic biosensor based on a metal-modified porous boron-doped diamond electrode, provided by the present invention, includes the following steps:

step 1: first, planting seed crystals on the surface of the silicon wafer substrate, and then, performing deposition on the surface of the substrate by hot wire chemical vapor deposition to obtain a boron-doped diamond film;

step 2: depositing a metal nickel layer on a surface of the boron-doped diamond film by magnetron sputtering;

step 3: performing thermal catalytic etching on the sample covered with the metal nickel layer, prepared in step 2, to form nickel particles embedded in the boron-doped diamond film;

step 4: performing anodic polarization treatment on the sample embedded with the nickel particles, prepared in step 3, by means of an electrochemical workstation to remove the metal nickel on the surface of the sample to form a porous structure;

step 5: depositing metal nanoparticles on the porous structure sample obtained in step 4 by electrodeposition by means of the electrochemical workstation, so as to obtain a metal-modified porous boron-doped diamond electrode; and

step 6: using the metal-modified porous boron-doped diamond electrode obtained in step 5 as a working electrode to assemble the nonenzymatic biosensor.

According to the method for preparing the nonenzymatic biosensor based on a metal-modified porous boron-doped diamond electrode, provided by the present invention, in step 1, the silicon wafer is a P-type heavily doped silicon wafer.

According to the method for preparing the nonenzymatic biosensor based on a metal-modified porous boron-doped diamond electrode, provided by the present invention, in step 1, a process of planting the seed crystals is as follows: the substrate is immersed in a suspension containing nanodiamond, ultrasonic vibration is performed for 30 min or longer, and finally cleaning and drying are performed.

In an actual operation process, first, the P-type heavily doped silicon wafer is placed in an acetone solution, ultrasonic cleaning is performed for 10 min to remove surface stains, and then, the P-type heavily doped silicon wafer is dried for later use.

According to the method for preparing the nonenzymatic biosensor based on a metal-modified porous boron-doped diamond electrode, provided by the present invention, in step 1, a technology of the hot wire chemical vapor deposition is as follows: a number of turns of a hot wire is 10-20, a temperature of the hot wire is 2,000-2,500° C., a mass flow ratio of gases introduced is hydrogen:methane:borane=49:1:(0.3-0.6), preferably 49:1:0.3, a growth pressure is 2.5-5 Kpa, a growth temperature is 700-900° C., and a growth time is 6-12 h.

According to the method for preparing the nonenzymatic biosensor based on a metal-modified porous boron-doped diamond electrode, provided by the present invention, in step 2, a technology of the magnetron sputtering is as follows: a nickel target with a purity 99.99% is used, a distance between the substrate and the target is 10-12 cm, an argon atmosphere is used, a deposition pressure is 0.5+/−0.05 Pa, a sputtering power is 50-150 W, a deposition time is 60 s, and a deposition thickness of the obtained nickel layer is 5-50 nm.

According to the method for preparing the nonenzymatic biosensor based on a metal-modified porous boron-doped diamond electrode, provided by the present invention, in step 3, a technology of the thermal catalytic etching is as follows: hydrogen is introduced for etching, a mass flow of the hydrogen is 40-100 SCCM, an etching temperature is 600-1,000° C., an etching pressure is controlled at 10-20 KPa, and an etching time is 100-300 min.

Under the above thermal catalytic conditions, nickel agglomerates into nanoparticles, and by means of defects in nickel lattices, short circuit diffusion occurs on interfaces and surfaces, so that carbon atoms in boron-doped diamond are continuously precipitated from the surfaces and interfaces of the nickel particles. Hydrogen in a tubular atmosphere annealing furnace reacts with the carbon atoms precipitated from the surfaces of the nickel particles at a high temperature to form hydrocarbon groups which leave the surfaces of the nickel particles, thereby ensuring dynamic conditions for the nickel particles to etch the boron-doped diamond. Finally, the nickel particles continuously penetrate into the boron-doped diamond to form a mosaic structure, and furthermore, an sp² phase is generated on the interface between the nickel particles and the boron-doped diamond.

According to the method for preparing the nonenzymatic biosensor based on a metal-modified porous boron-doped diamond electrode, provided by the present invention, in step 4, a process of the anodic polarization is as follows: first, the sample embedded with the nickel particles, prepared in step 3, is insulated and sealed, and then placed in a three-electrode system to connect to an electrochemical workstation, an anodic polarization voltage is +2.0+/−0.1 V, a polarization time is 150-180 s, and an electrolyte is a 1.0 M sodium sulfate solution

The nickel particles on the surface of the boron-doped diamond are removed by anodic polarization. The boron-doped diamond shows a porous structure, a specific surface area thereof as a carrier is greatly increased, and furthermore, the sp² phase generated on the interface can be retained, thereby improving the interface charge transfer.

According to the method for preparing the nonenzymatic biosensor based on a metal-modified porous boron-doped diamond electrode, provided by the present invention, in step 5, a technology of the electrodeposition of the metal nanoparticles is as follows: a deposition potential is −2.0 V to −1.2 V, a deposition time of each cycle is 30 s to 50 s, and a concentration of a deposition solution is 1 mM to 10 mM.

In the present invention, the type and content of metals can be accurately controlled by changing the deposition time and the number of potential transition cycles of different metals. The targeted performance adjustment and control of metal particle co-modified electrodes can be achieved for properties of different substances to be detected.

Preferably, when the metal nanoparticles are selected from gold and nickel nanoparticles, a number of deposition cycles is 5 respectively; first, the gold nanoparticles are deposited, a deposition potential is −1.0 V, a deposition time of one cycle is 30 s, and a deposition solution is a mM chloroauric acid solution; and then, the nickel nanoparticles are deposited, a deposition potential is −2.0 V, a deposition time of one cycle is 50 s, and a deposition solution is a 10 mM nickel nitrate solution.

Through the above technology, the sizes of gold-nickel composite nanoparticles are controlled to be 20-30 nm, and a gold-nickel content ratio is controlled to be 2:8. At this time, the best catalytic performance is obtained.

Preferably, when the metal nanoparticles are selected from gold and platinum nanoparticles, a number of deposition cycles is 4 respectively; first, the gold nanoparticles are deposited, a deposition potential is −1.0 V, a deposition time of one cycle is 50 s, and a deposition solution is a 1 mM chloroauric acid solution; and then, the platinum nanoparticles are deposited, a deposition potential of one cycle is −1.2 V, a deposition time of one cycle is 50 s, and a deposition solution is a 1 mM chloroplatinic acid solution.

Through the above technology, the sizes of gold-platinum composite nanoparticles are controlled to be 25-30 nm, and a gold-platinum content ratio is controlled to be about 2:8. At this time, the best catalytic performance is obtained.

Preferably, when the metal nanoparticles are selected from nickel and copper nanoparticles, a number of deposition cycles is 5 respectively; first, the nickel nanoparticles are deposited, a deposition potential is −2.0 V, a deposition time of one cycle is 50 s, and a deposition solution is a 10 mlvi nickel nitrate solution; and during the deposition of the copper nanoparticles, a deposition potential is −1.5 V, a deposition time of one cycle is 30 s, and a deposition solution is a 10 mM copper nitrate solution.

The sizes of nickel-copper composite nanoparticles obtained under this process are 20-30 nm, and a nickel-copper content ratio is 6:4. At this time, the best catalytic performance is obtained.

A porous boron-doped diamond substrate has a very large specific surface area, which can increase the load of the metal nanoparticles. Furthermore, the porous structure can ensure that the metal nanoparticles are anchored on the surface of the boron-doped diamond, thereby ultimately improving the sensitivity and stability of the electrode.

According to the method for preparing the nonenzymatic biosensor based on a metal-modified porous boron-doped diamond electrode, provided by the present invention, the metal-modified porous boron-doped diamond obtained in step (5) is used as a working electrode, a platinum electrode is used as a counter electrode, and an Ag/AgCl electrode is used as a contrast electrode to form a nonenzymatic sensor.

In the use of the nonenzymatic biosensor based on a metal-modified porous boron-doped diamond electrode, provided by the present invention, the nonenzymatic biosensor is used for detecting dopamine or glucose. Preferably, the nonenzymatic biosensor is used for detecting glucose.

The present invention provides a nonenzymatic biosensor based on a metal-modified porous boron-doped diamond electrode. A working electrode of the nonenzymatic biosensor is a metal-modified porous boron-doped diamond electrode. The electrode working layer is a porous boron-doped diamond layer, the surface of which is modified with metal nanoparticles. A pore surface of the porous boron-doped diamond layer contains an sp² phase. The inventors found that by retaining the generated sp² phase on the pore surface of the porous boron-doped diamond layer, on the one hand, the interface charge transfer can be improved, and a faster charge transfer rate means a higher electrochemical reaction rate, so the detection sensitivity and linear range of the sensor can be increased; and on the other hand, the adhesion with the metal nanoparticles can be increased, thereby further improving the stability of the electrode.

According to the method provided by the present invention, by means of anodic polarization treatment, nickel particles can be removed in a controlled manner to form a porous structure, and furthermore, the sp² phase generated on the interface is retained. In addition, in the present invention, the type and content of metals can be accurately controlled by changing the deposition time and the number of potential transition cycles of different metals, and the targeted performance adjustment and control of metal particle co-modified electrodes can be achieved for properties of different substances to be detected, thereby obtaining the best detection performance.

### BRIEF DESCRIPTION OF THE DRAWINGS

FIG. 1 shows a scanning electron microscope (SEM) diagram of a diamond film not etched in Example 1.

FIG. 2 shows an SEM diagram of a porous morphology of the diamond film etched in Example 1.

FIG. 3 shows CV detection curves of a metal-modified porous boron-doped diamond electrode modified with gold and platinum in different ratios in Comparative Example 2, where the ratio in a curve 1 is 1:9, the ratio in a curve 2 is 3:7, the ratio in a curve 4 is 1:1, and the ratio in a curve 3 is 3:2.

FIG. 4 shows an SEM diagram of a morphology of Ni nanoparticles modified with diamond not etched in Comparative Example 3.

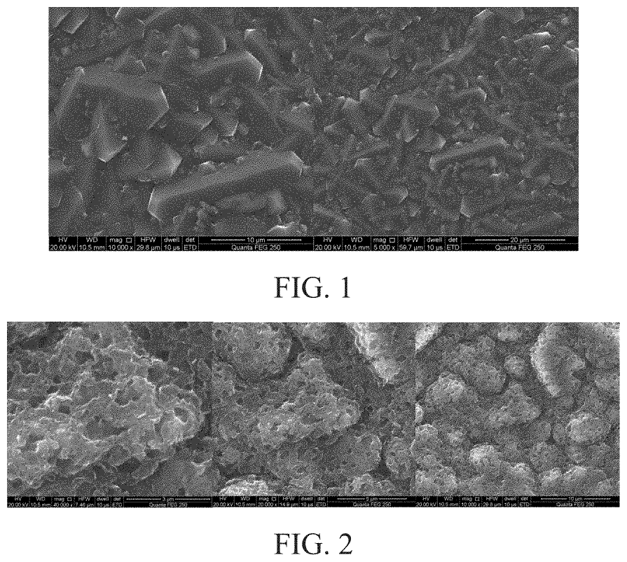

### DETAILED DESCRIPTION OF THE EMBODIMENTS

The substantive features and significant progress of the present invention are further illustrated through the following embodiments, but the present invention is by no means limited to the embodiments.

### Example 1

Step 1: Preparation of a boron-doped diamond film. First, a silicon wafer substrate was placed in an acetone solution. Ultrasonic cleaning was performed for 5-20 min to remove oil stains on a surface. Then, ultrasonic cleaning was performed in deionized water for 10-20 min. The silicon wafer substrate was dried in a drying furnace and then put into a chemical vapor deposition chamber for the growth of boron-doped diamond. During the growth process, the number of turns of a hot wire was 10-20. A temperature of the hot wire was controlled at 2,000-2,500° C. A surface temperature of the substrate was 700-900° C. A gas ratio was methane:borane:hydrogen=1:49:0.3. A cavity pressure was about 2.5-5 Kpa. A grain size of the grown diamond film was 5-10 μm in diameter. A film thickness was 5-20 μm.

Step 2: Nickel layer sputtering. A method was as follows. A physical magnetron sputtering device was used. Under an atmospheric pressure of 0.5-2 Pa, a layer of nickel film was uniformly sputtered on the diamond film in step 1 by a high-purity nickel target with a purity of 99.99%. A sputtering power was 50-150 W. A thickness of the nickel layer was 5-50 nm.

Step 3: High-temperature heat treatment etching in a hydrogen environment. A method was as follows. A sheet prepared in step 2 was put into a cold-wall heat treatment furnace. 40-100 SCCM hydrogen was introduced. An etching temperature was controlled at 600-1,000° C. An etching pressure was controlled at 10-20 Kpa. An etching time was 100-300 min.

Step 4: Removal of nickel particles. A method was as follows. The sample obtained in step 3 was encapsulated and then placed in an electrochemical workstation for anodic polarization. An anodic polarization voltage was +2.0 V. A polarization time was 180 s. An electrolyte was a 1.0 M sodium sulfate solution.

Step 5: Co-modification of gold and nickel nanoparticles. Metal nanoparticles were deposited by a square wave transition potential. The number of deposition cycles was 5, During the deposition of gold nanoparticles, a deposition potential was −1.0 V. A deposition time of one cycle was 30 s. A deposition solution was a 1 mM chloroauric acid solution, During the deposition of nickel nanoparticles, a deposition potential was −2.0 V. A deposition time of one cycle was 50 s. A deposition solution was a 10 mM nickel nitrate solution. The sizes of gold-nickel composite nanoparticles obtained under this process were 20-30 nm, and a gold-nickel content ratio was about 2:8. At this time, the best catalytic performance was obtained.

Step 6: Preparation of a sensor. A method was as follows. After the electrode obtained in step 5 was encapsulated, a reference electrode and a counter electrode were used together with the encapsulated electrode to form a three-electrode detection sensor.

The three-electrode detection sensor obtained in Example 1 was used for detecting glucose concentration. The sensitivity was 1,586 μAcm⁻²mM⁻¹. A linear range was 0.001-30 mM. A detection limit was 0.0005 mM. During the 30-day cycle stability test, only 7% of the response current was lost.

### Example 2

Step 1: Preparation of a boron-doped diamond film. First, a silicon wafer substrate was placed in an acetone solution. Ultrasonic cleaning Was performed for 10 min to remove oil stains on a surface. Then, ultrasonic cleaning was performed in deionized water for 15 min. The silicon wafer substrate was dried in a drying furnace and then put into a chemical vapor deposition chamber for the growth of boron-doped diamond. During the growth process, the number of turns of a hot wire was 15. A temperature of the hot wire was controlled at 2,250° C. A surface temperature of the substrate was 800° C. A gas ratio was methane:borane:hydrogen=1:49:0.3. A cavity pressure was about 3.0 Kpa. A grain size of the grown diamond film was 6-8 μm in diameter. A film thickness was 10-15 μm.

Step 2: Nickel layer sputtering. A method was as follows. A physical magnetron sputtering device was used. Under an atmospheric pressure of 1 Pa, a layer of nickel film was uniformly sputtered on the diamond film in step 1 by a high-purity nickel target with a purity of 99.99%. A sputtering power was 100 W. A thickness of the nickel layer was 20-40 nm.

Step 3: High-temperature heat treatment etching in a hydrogen environment. A method was as follows. A sheet prepared in step 2 was put into a cold-wall heat treatment furnace. 60 SCCM hydrogen was introduced. An etching temperature was controlled at 800° C. An etching pressure was controlled at 15 Kpa. An etching time was 200 min.

Step 4: Removal of nickel particles. A method was as follows. The sample obtained in step 3 was encapsulated and then placed in an electrochemical workstation for anodic polarization. An anodic polarization voltage was ±2.0 V. A polarization time was 180 s. An electrolyte was a 1.0 M sodium sulfate solution.

Step 5: Co-modification of gold and platinum nanoparticles. Metal nanoparticles were deposited by a square wave transition potential. The number of deposition cycles was 4. During the deposition of gold nanoparticles, a deposition potential was −1.0 V. A deposition time was 50 s. A deposition solution was a 1 mM chloroauric acid solution. During the deposition of platinum nanoparticles, a deposition potential was −1.2 V. A deposition time of one cycle was 50 s. A deposition solution was a 1 mM chloroplatinic acid solution. The sizes of gold-platinum composite nanoparticles obtained under this process were 25-30 nm, and a gold-platinum content ratio was about 1:1. At this time, the best catalytic performance was obtained.

Step 6: Preparation of a sensor. A method was as follows. After the electrode obtained in step 5 was encapsulated, a reference electrode and a counter electrode were used together with the encapsulated electrode to form a three-electrode detection sensor for detecting dopamine concentration. The sensitivity was 208 μAcm⁻²mM⁻¹. A detection limit was 0.07 μM.

### Example 3

Step 1: Preparation of a boron-doped diamond film. First, a silicon wafer substrate was placed in an acetone solution. Ultrasonic cleaning was performed for 10 min to remove oil stains on a surface. Then, ultrasonic cleaning was performed in deionized water for 15 min. The silicon wafer substrate was dried in a drying furnace and then put into a chemical vapor deposition chamber for the growth of boron-doped diamond. During the growth process, the number of turns of a hot wire was 15. A temperature of the hot wire was controlled at 2,250° C. A surface temperature of the substrate was 800° C. A gas ratio was methane:borane:hydrogen=1:49:0.3. A cavity pressure was about 3.0 Kpa. A grain size of the grown diamond film was 6-8 μm is diameter. A film thickness was 10-15 μm.

Step 2: Nickel layer sputtering. A method was as follows. A physical magnetron sputtering device was used. Under an atmospheric pressure of 1 Pa, a layer of nickel film was uniformly sputtered on the diamond film in step 1 by a high-purity nickel target with a purity of 99.99%. A sputtering power was 100 W. A thickness of the nickel layer was 20-40 μm.

Step 3: High-temperature heat treatment etching in a hydrogen environment. A method was as follows. A sheet prepared in step 2 was put into a cold-wall heat treatment furnace. 60 SCCM hydrogen was introduced. An etching temperature was controlled at 800° C. An etching pressure was controlled at 15 Kpa. An etching time was 200 min.

Step 4: Removal of nickel particles. A method was as follows. The sample obtained in step 3 was encapsulated and then placed in an electrochemical workstation for anodic polarization. An anodic polarization voltage was +2.0 V. A polarization time was 180 s. An electrolyte was a 1.0 M sodium sulfate solution.

Step 5: Co-modification of nickel and copper nanoparticies. Metal nanoparticles were deposited by a square wave transition potential. The number of deposition cycles was 5. During the deposition of nickel nanoparticles, a deposition potential was −2.0 V. A deposition time was 50 s. A deposition solution was a 10 mM nickel nitrate solution. During the deposition of copper nanoparticles, a deposition potential was −1.5 V. A deposition time of one cycle was 30 s. A deposition solution was a 10 mM copper nitrate solution. The sizes of nickel-copper composite nanoparticles obtained under this process were 20-30 nm, and a nickel-copper content ratio was about 5:4. At this time, the best catalytic performance was obtained.

Step 6: Preparation of a sensor. A method was as follows, After the electrode obtained in step 5 was encapsulated, a reference electrode and a counter electrode were used together with the encapsulated electrode to form a three-electrode detection sensor.

The three-electrode detection sensor obtained in Example 3 was used for detecting glucose concentration. The sensitivity was 1,730 μAcm⁻²mM⁻1. A linear range was 0.02-8.5 mM. A detection limit was 0.005 mM. During the 30-day cycle stability test, only 5% of the response current was lost.

### Comparative Example 1

Other conditions were the same as those in Example 1, except that nitric acid was used for removing nickel particles. As a result, the sp² phase on the interface was removed, and then, a result of Comparative Example 1 for detecting glucose concentration was given.

The sensitivity of the electrode was only 566 μAcm⁻²mM⁻¹. A linear range was 0.01-3.87 mM. A detection limit was 0.008 mM. During the 30-day stability test, more than 80% of the response current was lost.

### Comparative Example 2

Other conditions were the same as those in Example 2, except that deposition parameters of Pt were changed by fixing deposition parameters of Au unchanged. Deposition times of one cycle were designed as 5 s, 25 s, 50 s and 75 s. Four electrodes were obtained. Through elemental analysis, Pt:Au of the four electrodes was 1:9, 3:7, 1:1 and 3:2 respectively. The electrode, Pt:Au of which was 1:1, was the electrode in Example 2.

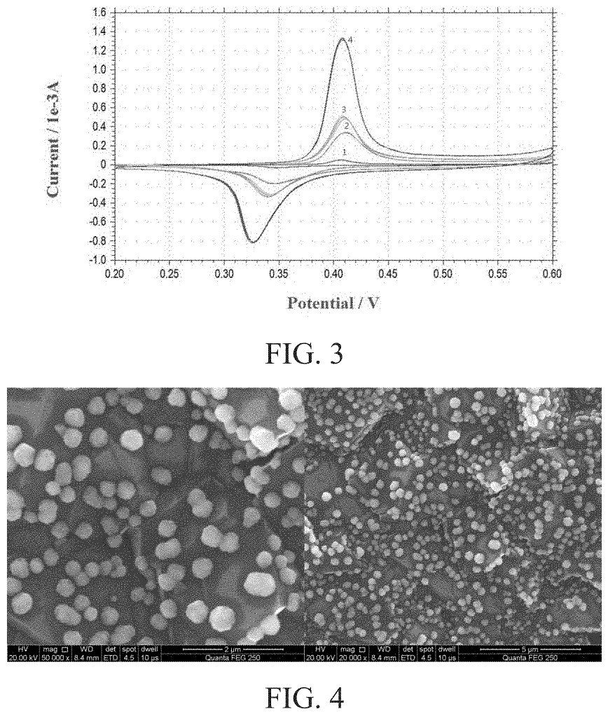

CV detection curves of the 4 electrodes, Pt:Au of which was 1:9, 3:7, 1:1 and 3:2 respectively, in a mixed solution of 0.5 M NaOH and 1 mM glucose were shown in FIG. 3. The 4 electrodes corresponded to curves 1, 2, 4 and 3 respectively. A scanning potential range was 0.2-0.6 V, and a scanning rate was 50 mVs⁻¹. It can be seen from the figure that when Pt:Au is 1:1 (curve 4), the catalytic activity of the obtained BDD is the highest.

### Comparative Example 3

Other conditions were the same as those in Example 3, except that no etching was performed for forming a porous structure, that is, the metal nickel was repaired. Results (as shown in FIG. 4) show that the metal nickel can be successfully repaired, but the electrode was unstable due to falling off during the use.

What is claimed is:

1. A nonenzymatic biosensor based on a metal-modified porous boron-doped diamond electrode, wherein a working electrode of the nonenzymatic biosensor is the metal-modified porous boron-doped diamond electrode comprising a silicon wafer substrate and an electrode working layer; and the electrode working layer is arranged on a surface of the silicon wafer substrate, the electrode working layer is a porous boron-doped diamond layer with a surface modified with metal nanoparticles, and a pore surface of the porous boron-doped diamond layer comprises an sp² phase.

2. The nonenzymatic biosensor based on the metal-modified porous boron-doped diamond electrode according to claim 1, wherein a thickness of the porous boron-doped diamond layer is 5 μm-20 μm, a grain size is 5 μm-20 μm, and a crystal surface (111) is an exposed surface.

3. The nonenzymatic biosensor based on the metal-modified porous boron-doped diamond electrode according to claim 1, wherein particle sizes of the metal nanoparticles are 20 nm-30 nm; and the metal nanoparticles are selected from at least one of gold nanoparticles, platinum nanoparticles, nickel nanoparticles, and copper nanoparticles.

4. The nonenzymatic biosensor based on the metal-modified porous boron-doped diamond electrode according to claim 3, wherein the metal nanoparticles are selected from the gold nanoparticles and the nickel nanoparticles, and according to an atomic ratio, gold:nickel=2:8; the metal nanoparticles are selected from the gold nanoparticles and the platinum nanoparticles, and according to an atomic ratio, gold:platinum=1:1; and the metal nanoparticles are selected from the nickel nanoparticles and the copper nanoparticles, and according to an atomic ratio, nickel:copper=6:4.

5. A method for preparing the nonenzymatic biosensor based on the metal-modified porous boron-doped diamond electrode according to claim 1, comprising the following steps:
step 1: first, planting seed crystals on the surface of the silicon wafer substrate, and then, performing a deposition on the surface of the silicon wafer substrate by a hot wire chemical vapor deposition to obtain a boron-doped diamond film;
step 2: depositing a metal nickel layer on a surface of the boron-doped diamond film by a magnetron sputtering;
step 3: performing a thermal catalytic etching on a sample covered with the metal nickel layer prepared in the step 2 to form nickel particles embedded in the boron-doped diamond film;
step 4: performing an anodic polarization treatment on the sample embedded with the nickel particles prepared in the step 3 by an electrochemical workstation to remove metal nickel on a surface of the sample to form a porous structure;
step 5: depositing the metal nanoparticles on the porous structure of the sample obtained in the step 4 by an electrodeposition by the electrochemical workstation to obtain the metal-modified porous boron-doped diamond electrode; and
step 6: using the metal-modified porous boron-doped diamond electrode obtained in the step 5 as the working electrode to assemble the nonenzymatic biosensor.

6. The method for preparing the nonenzymatic biosensor based on the metal-modified porous boron-doped diamond electrode according to claim 5, wherein
in the step 1, a process of planting the seed crystals is as follows: the silicon wafer substrate is immersed in a suspension containing nanodiamonds, an ultrasonic vibration is performed for 30 min or longer, and finally, cleaning and drying are performed; and
in the step 1, a technology of the hot wire chemical vapor deposition is as follows: a number of turns of a hot wire is 10-20, a temperature of the hot wire is 2,000° C.-2,500° C., a mass flow ratio of gases introduced is hydrogen:methane:borane=49:1:(0.3-0.6), a growth pressure is 2.5 Kpa-5 Kpa, a growth temperature is 700° C.-900° C., and a growth time is 6 h-12 h.

7. The method for preparing the nonenzymatic biosensor based on the metal-modified porous boron-doped diamond electrode according to claim 5, wherein
in the step 2, a technology of the magnetron sputtering is as follows: a nickel target with a purity≥99.99% is used, a distance between the silicon wafer substrate and the nickel target is 10 cm-12 cm, an argon atmosphere is used, a deposition pressure is 0.5+/−0.05 Pa, a sputtering power is 50 W-150 W, a deposition time is 60 s, and a deposition thickness of the metal nickel layer is 5 nm-50 nm; and
in the step 3, a technology of the thermal catalytic etching is as follows: hydrogen is introduced for an etching, a mass flow of the hydrogen is 40 SCCM-100 SCCM, an etching temperature is 600° C.-1,000° C., an etching pressure is controlled at 10 KPa-20 KPa, and an etching time is 100 min-300 min.

8. The method for preparing the nonenzymatic biosensor based on the metal-modified porous boron-doped diamond electrode according to claim 5, wherein
in the step 4, a process of the anodic polarization is as follows: first, the sample embedded with the nickel particles prepared in the step 3 is insulated and sealed, and then placed in a three-electrode system to connect to the electrochemical workstation, an anodic polarization voltage is +2.0+/−0.1 V, a polarization time is 150 s-180 s, and an electrolyte is a 1.0 M sodium sulfate solution; and
in the step 5, a technology of the electrodeposition of the metal nanoparticles is as follows: a deposition potential is −2.0 V to −1.2 V, a deposition time of each cycle is 30 s to 50 s, and a concentration of a deposition solution is 1 mM to 10 mM.

9. The method for preparing the nonenzymatic biosensor based on the metal-modified porous boron-doped diamond electrode according to claim 8, wherein
when the metal nanoparticles are selected from gold nanoparticles and nickel nanoparticles, a number of deposition cycles is 5 respectively; first, the gold nanoparticles are deposited, a deposition potential is −1.0 V, a deposition time of one cycle is 30 s, and a deposition solution is a 1 mM chloroauric acid solution; and then, the nickel nanoparticles are deposited, a deposition potential is −2.0 V, a deposition time of one cycle is 50 s, and a deposition solution is a 10 mM nickel nitrate solution;
when the metal nanoparticles are selected from gold nanoparticles and platinum nanoparticles, a number of deposition cycles is 4 respectively; first, the gold nanoparticles are deposited, a deposition potential is −1.0 V a deposition time of one cycle is 50 s, and a deposition solution is a 1 mM chloroauric acid solution; and then, the platinum nanoparticles are deposited, a deposition potential of one cycle is −1.2 V, a deposition time of one cycle is 50 s, and a deposition solution is a 1 mM chloroplatinic acid solution; and
when the metal nanoparticles are selected from nickel nanoparticles and copper nanoparticles, a number of deposition cycles is 5 respectively; first, the nickel nanoparticies are deposited, a deposition potential is −2.0 V, a deposition time of one cycle is 50 s, and a deposition solution is a 10 nM nickel nitrate solution; and during a deposition of the copper nanoparticles, a deposition potential is −1.5 V, a deposition time of one cycle is 30 s, and a deposition solution is a 10 mM copper nitrate solution.

10. A method of a use of the nonenzymatic biosensor based on the metal-modified porous boron-doped diamond electrode according to claim 1, wherein the nonenzymatic biosensor is used for detecting dopamine or glucose.

11. The method of the use of the nonenzymatic biosensor based on the metal-modified porous boron-doped diamond electrode according to claim 10, wherein in the nonenzymatic biosensor based on the metal-modified porous boron-doped diamond electrode, a thickness of the porous boron-doped diamond layer is 5 μm-20 μm, a grain size is 5 μm-20 μm, and a crystal surface (111) is an exposed surface.

12. The method of the use of the nonenzymatic biosensor based on the metal-modified porous boron-doped diamond electrode according to claim 10, wherein in the nonenzymatic biosensor based on the metal-modified porous boron-doped diamond electrode, particle sizes of the metal nanoparticles are 20 nm-30 nm; and the metal nanoparticles are selected from at least one of gold nanoparticles, platinum nanoparticles, nickel nanoparticles, and copper nanoparticles.

13. The method of the use of the nonenzymatic biosensor based on the metal-modified porous boron-doped diamond electrode according to claim 12, wherein in the nonenzymatic biosensor based on the metal-modified porous boron-doped diamond electrode, the metal nanoparticles are selected from the gold nanoparticles and the nickel nanoparticles, and according to an atomic ratio, gold:nickel=2:8; the metal nanoparticles are selected from the gold nanoparticles and the platinum nanoparticles, and according to an atomic ratio, gold:platinum=1:1; and the metal nanoparticles are selected from the nickel nanoparticles and the copper nanoparticles, and according to an atomic ratio, nickel:copper=6:4.

14. The method for preparing the nonenzymatic biosensor based on the metal-modified porous boron-doped diamond electrode according to claim 5, wherein in the nonenzymatic biosensor based on the metal-modified porous boron-doped diamond electrode, a thickness of the porous boron-doped diamond layer is 5 μm-20 μm, a grain size is 5 μm-20 μm, and a crystal surface (111) is an exposed surface.

15. The method for preparing the nonenzymatic biosensor based on the metal-modified porous boron-doped diamond electrode according to claim 5, wherein in the nonenzymatic biosensor based on the metal-modified porous boron-doped diamond electrode, particle sizes of the metal nanoparticles are 20 nm-30 nm; and the metal nanoparticles are selected from at least one of gold nanoparticles, platinum nanoparticles, nickel nanoparticles, and copper nanoparticles.

16. The method for preparing the nonenzymatic biosensor based on the metal-modified porous boron-doped diamond electrode according to claim 15, wherein in the nonenzymatic biosensor based on the metal-modified porous boron-doped diamond electrode, the metal nanoparticles are selected from the gold nanoparticles and the nickel nanoparticles, and according to an atomic ratio, gold:nickel=2:8; the metal nanoparticles are selected from the gold nanoparticles and the platinum nanoparticles, and according to an atomic ratio, gold:platinum=1:1; and the metal nanoparticles are selected from the nickel nanoparticles and the copper nanoparticles, and according to an atomic ratio, nickel:copper=6:4.
